(19) United States
(12) Patent Application Publication
(10) Pub. No.: US 2025/0177602 A1
(43) Pub. Date: Jun. 5, 2025

(54) ECO-FRIENDLY ADHESIVE CONTAINING LIGNIN HYDROGEL AND METHOD FOR PRODUCING ECO- FRIENDLY LIGNIN HYDROGEL

(71) Applicant: POSTECH RESEARCH AND BUSINESS DEVELOPMENT FOUNDATION, Pohang-si (KR)

(72) Inventors: Youn Soo KIM, Pohang-si (KR); YiLuo HU, Pohang-si (KR); Jae Won CHOI, Pohang-si (KR); Dong Soo HWANG, Pohang-si (KR); Asila Ahmed Mohmmed OSMAN, Pohang-si (KR)

(21) Appl. No.: 18/926,523

(22) Filed: Oct. 25, 2024

(30) Foreign Application Priority Data
Dec. 5, 2023 (KR) 10-2023-0174734
Jun. 25, 2024 (KR) 10-2024-0082961

(51) Int. Cl. A61L 24/00

(52) U.S. Cl. CPC A61L 24/0094; A61L 24/0031

(57) ABSTRACT

An object of the invention is to provide an eco-friendly adhesive which is eco-friendly and economical using lignin hydrogel. In order to achieve the above-identified object, in an eco-friendly adhesive including lignin hydrogel according to the invention, the lignin hydrogel includes a network structure through a bonding between lignin and a zwitterionic polymer.

### BACKGROUND OF THE INVENTION

### 1. Field of the Invention

The present invention relates to an eco-friendly adhesive including lignin hydrogel, and particularly, lignin hydrogel is characterized by including a network structure through a between a lignin-based material and a zwitterionic polymer.

### 2. Description of the Related Art

In recent years, the development of an eco-friendly adhesive has been active. An eco-friendly adhesive is an adhesive designed to minimize negative impacts on the environment and human body, and can be applied to various fields such as cosmetics, food packaging, and biodegradable adhesives.

In particular, among eco-friendly adhesives, a bioadhesive refers to an adhesive that can attach to biological tissue. It is widely used for medical purposes and can be utilized in fields such as wound healing, tissue regeneration, and drug delivery. As materials for bioadhesives, protein-based, polysaccharide-based, and synthetic polymer-based materials are mainly used, and properties such as biocompatibility, adhesive strength, biodegradability, and flexibility are required.

Meanwhile, lignin is one of the three major components of a plant cell wall and is the second most abundant plant polymer on earth. Lignin is not only abundant in nature, but is also an important by-product of the pulp and paper industry. Lignin produced in this way amounts to about 50 million tons per year, but most of it is discarded or used as fuel.

In recent years, in order to utilize the environmental and economical values of lignin, which has biocompatibility, eco-friendliness, and low toxicity, studies have been conducted on production methods for high value-added functional materials such as bioplastics, adhesives, and dispersants. However, due to the irregular structure of lignin and its poor miscibility with other materials, it is still hardly applied in practical industry.

### SUMMARY OF THE INVENTION

An object of the invention is to provide an eco-friendly and economical adhesive using lignin hydrogel.

In addition, another object of the invention is to provide a method for producing lignin hydrogel for an eco-friendly adhesive, which can easily and economically produce lignin hydrogel on a large scale.

In order to achieve the above-identified objects, in the eco-friendly adhesive including lignin hydrogel according to the invention, the lignin hydrogel includes a network structure through a bonding between a lignin material and a zwitterionic polymer.

Additionally, in the eco-friendly adhesive including lignin hydrogel according to an embodiment of the invention, the zwitterionic polymer may be any one among 2-(methacryloyloxy)-3-(trimethylammonio) propane-1-sulfonate, 3-pyridinio-2-methacryloyloxypropane-1-sulfonate, arginine methacrylate, 3-N-methylpyrrolidinio-2-methacryloyloxypropane-1-sulfonate, poly(carboxybetaine) based on vinylimidazole, and poly(sulfobetaine) based on vinylimidazole.

Additionally, in the eco-friendly adhesive including lignin hydrogel according to an embodiment of the invention, the zwitterionic polymer may be physically cross-linked by a cation-m interaction and a dipole-dipole interaction on the surface of the lignin.

Additionally, in the eco-friendly adhesive including lignin hydrogel according to an embodiment of the invention, when the total weight of the lignin hydrogel is 100 parts by weight, the sum of the weights of the lignin-based material and the zwitterionic polymer may be 10-70 parts by weight.

Additionally, in the eco-friendly adhesive including lignin hydrogel according to an embodiment of the invention, when the total weight of the lignin hydrogel is 100 parts by weight, the sum of the weights of the lignin-based material and the zwitterionic polymer may be 20-50 parts by weight.

Additionally, in the eco-friendly adhesive including lignin hydrogel according to an embodiment of the invention, when the total weight of the lignin hydrogel is 100 parts by weight, the sum of the weights of the lignin-based material and the zwitterionic polymer may be 10-20 parts by weight, and the lignin hydrogel may have a viscoelastic property.

Additionally, the eco-friendly adhesive including lignin hydrogel according to an embodiment of the invention may be a bioadhesive.

Additionally, the eco-friendly adhesive including lignin hydrogel according to an embodiment of the invention may be a bioadhesive included in a bioimplant device.

According to the invention, there may be provided a method for preparing lignin hydrogel for an eco-friendly adhesive, which includes adding lignin and a zwitterionic monomer and a polymerization initiator to water and mixing to prepare a mixture; and irradiating a microwave to the mixture.

Additionally, in the method for preparing lignin hydrogel for an eco-friendly adhesive according to an embodiment of the invention, the zwitterionic monomer may be imidazolium sulfobetaine.

Additionally, in the method for preparing lignin hydrogel for an eco-friendly adhesive according to an embodiment of the invention, the polymerization initiator may be 2,2′-azobis(2-methylpropionamidine) dihydrochloride.

Additionally, in the method for preparing lignin hydrogel for an eco-friendly adhesive according to an embodiment of the invention, when the total weight of said mixture is 100 parts by weight, the sum of the weights of the lignin-based material and the zwitterionic polymer may be 10-70 parts by weight.

Additionally, in the method for preparing lignin hydrogel for an eco-friendly adhesive according to an embodiment of the invention, the microwave irradiation may be performed so that the temperature of the mixture can be 60° C. or higher and maintained thereat for 1 hour or more.

### BRIEF DESCRIPTION OF THE DRAWINGS

FIG. 1 is a view illustrating the structural formulas of zwitterionic polymers of lignin hydrogel according to an embodiment of the invention;

FIG. 2 is a schematic diagram illustrating a process for preparing lignin hydrogel according to an embodiment of the invention;

FIG. 3 shows images and a graph illustrating evaluation results of mechanical properties of lignin hydrogel according to an embodiment of the invention;

FIG. 4 shows images illustrating the results of evaluating the self-healing ability of lignin hydrogel according to an embodiment of the present invention;

FIG. 5 shows graphs illustrating evaluation results of adhesive property of lignin hydrogel according to an embodiment of the invention;

FIG. 6 shows images and a graph illustrating evaluation results of adhesive property of lignin hydrogel for various subjects according to an embodiment of the invention;

FIG. 7 shows images and graphs illustrating evaluation results of biocompatibility of lignin hydrogel according to an embodiment of the invention; and

FIG. 8 shows a graph illustrating evaluation results of conductivity of lignin hydrogel according to an embodiment of the invention.

### DETAILED DESCRIPTION OF THE EXEMPLARY EMBODIMENTS

Hereinbelow, embodiments of the invention are described in detail with reference to the attached drawings so that those having ordinary knowledge in the technical field to which the invention pertains can easily implement the invention. However, the invention can be implemented in various forms and is not limited to the embodiments described herein.

Additionally, in order to clearly explain the invention in the drawings, parts that are not related to the explanation are omitted, and similar parts are given similar reference numerals throughout the specification.

The eco-friendly adhesive according to the invention may include lignin hydrogel including a network structure through a bonding between lignin and a zwitterionic polymer.

As described above, lignin has the characteristics of biocompatibility and eco-friendliness, and thus the hydrogel using lignin is suitable as an eco-friendly adhesive. However, there is a problem in that it is difficult to hydrogelize lignin due to low dispersibility thereof. In the invention, the low dispersibility of lignin was overcome by forming a network structure through bonding with a zwitterionic polymer.

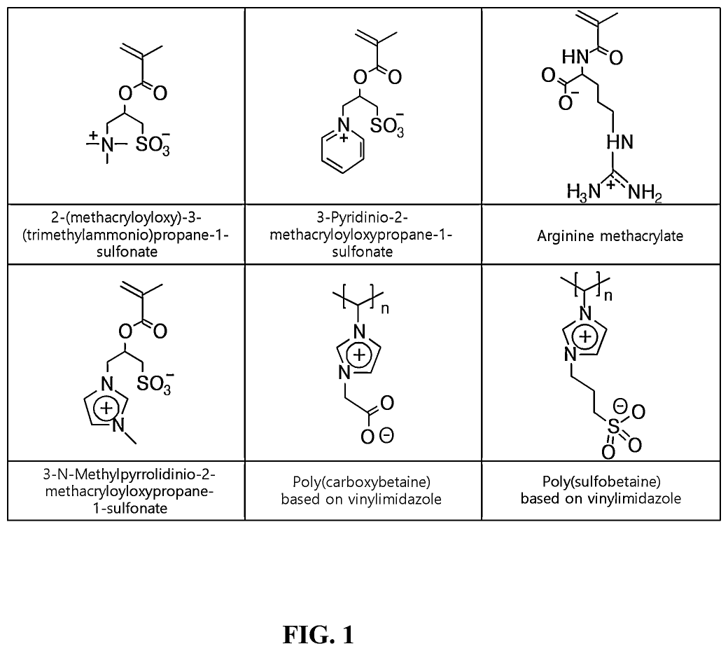

In this lignin hydrogel, the zwitterionic polymer may be any one among 2-(methacryloyloxy)-3-(trimethylammonio) propane-1-sulfonate, 3-pyridinio-2-methacryloyloxypropane-1-sulfonate, arginine methacrylate, 3-N-methylpyrrolidinio-2-methacryloyloxypropane-1-sulfonate, poly(carboxybetaine) based on vinylimidazole, and poly(sulfobetaine) based on vinylimidazole. The structural formula of this polymer is shown in FIG. 1.

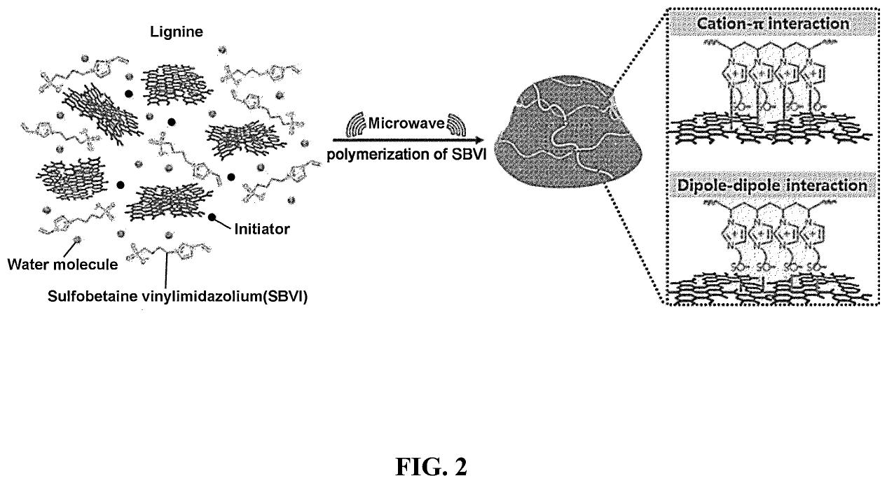

Such a zwitterionic polymer is physically cross-linked by a cation-T interaction and a dipole-dipole interaction on the surface of a lignin-based material, which includes either one or both of lignin and lignocellulose, thereby forming lignin hydrogel (see FIG. 2).

In this lignin hydrogel, when the total weight of the lignin hydrogel is 100 parts by weight, the sum of the weights of the lignin-based material and the polymer is preferably 10-70 parts by weight, and more preferably 20-50 parts by weight. When the combined content of the lignin-based material and the polymer is too low or too high, it becomes difficult to form a network structure for the hydrogel.

In particular, when the combined content of the lignin-based material and the polymer is 10-20 parts by weight, the hydrogel will have a viscoelastic property, and the hydrogel will have such a viscoelastic property even when the conductivity and mechanical properties are inferior to those of hydrogels including a high content of the lignin-based material, thereby enabling the preparation of an eco-friendly adhesive using these properties.

The lignin hydrogel according to the invention may be an eco-friendly adhesive that can be used in various fields. In particular, since lignin hydrogel has the characteristics that it is non-toxic to the human body and has high bioadhesiveness, it can be very advantageous for use as a bioadhesive for wound treatment, etc.

The method for preparing this lignin hydrogel may include the steps of adding a lignin-based material, a zwitterionic monomer, and a polymerization initiator to water and mixing them to prepare a mixture; and irradiating a microwave to the mixture.

In particular, the zwitterionic monomer may be imidazolium sulfobetaine, and through which the lignin hydrogel may easily be formed using microwave irradiation.

In addition, the polymerization initiator may be 2,2′-azobis(2-methylpropionamidine) dihydrochloride.

Meanwhile, in the method for preparing lignin hydrogel, when the total weight of the mixture is 100 parts by weight, the sum of the weights of the lignin-based material and the zwitterionic polymer may be included in an amount of 10-70 parts by weight. When the content of the lignin-based material is too low or too high, it becomes difficult to form a hydrogel smoothly.

In addition, the microwave irradiation may be performed so that the temperature of the mixture can be 60° C. or higher and maintained thereat for 1 hour or more.

### EXAMPLES

For the formation of lignin hydrogel, lignin, SBVI monomers, and a V-50 initiator (0.05 mmol) were dispersed in 15 mL of deionized water to prepare a mixture, which was then transferred to a microwave reaction vessel, tightly sealed, and irradiated with a microwave. The reaction was heated to 80° C. within 30 minutes and then maintained at 80° C. for 2 hours.

### Preparation of Materials

Lignin, 2,2′-azobis(2-methylpropionamidine) dihydrochlorid (initiator, Sigma-Aldrich, V-50), and imidazolium sulfobetaine (SBVI) were prepared.

### Synthesis of Lignin Hydrogel Via Microwave Synthesis

Lignin (0.1 g, 0.2 g, 0.3 g, 0.4 g, and 0.5 g), SBVI monomers (1.4 g, 2.8 g, 4.2 g, 5.6 g, and 7.0 g), and a V-50 initiator (0.05 mmol) were dispersed in 15 mL of deionized water. The dispersed mixture was transferred to a microwave reaction vessel, tightly sealed, and the reaction was started. The reaction was heated to 80° C. within 30 minutes and maintained at 80° C. for 2 hours. The samples of the thus-prepared lignin hydrogel were named according to the contents of lignin and SBVI, as shown in Table 1 below.

| Sample Name | Lig1 | Lig2 | Lig3 | Lig4 | Lig5 |
|---|---|---|---|---|---|
| Lignin (g) | 0.1 | 0.2 | 0.3 | 0.4 | 0.5 |
| SBVI (g) | 1.4 | 2.8 | 4.2 | 5.6 | 7.0 |
| w/v % | 10% | 20% | 30% | 40% | 50% |

### Mechanical Properties

The mechanical properties of lignin hydrogel were measured using a rheometer (trade name: T-1040, manufactured by TA instrument) in parallel plates with a diameter of 20 mm. The elastic modulus of the hydrogel was measured at frequencies of 1-100 rad/s at 1% strain.

### Test of Conductivity

The conductivity of lignin hydrogel was confirmed by two-electrode EIS (trade name: WizEIS-1200 premium, manufactured by WizMAC). The hydrogel was placed between aluminum foil with a 1 mm m silicone mold to block the contact between the two electrodes, and frequency sweeps were measured from 0.1 Hz to 106 Hz.

### Test of Adhesive Property

The adhesive property of lignin hydrogel was confirmed by a lap shear test method using a universal testing machine (trade name: INSTRON 5848, manufactured by INSTRON). The lignin hydrogel was evenly spread between two pig skins (10 mm×10 mm×1 mm) and measured at room temperature at a shear rate of 10 mm/min.

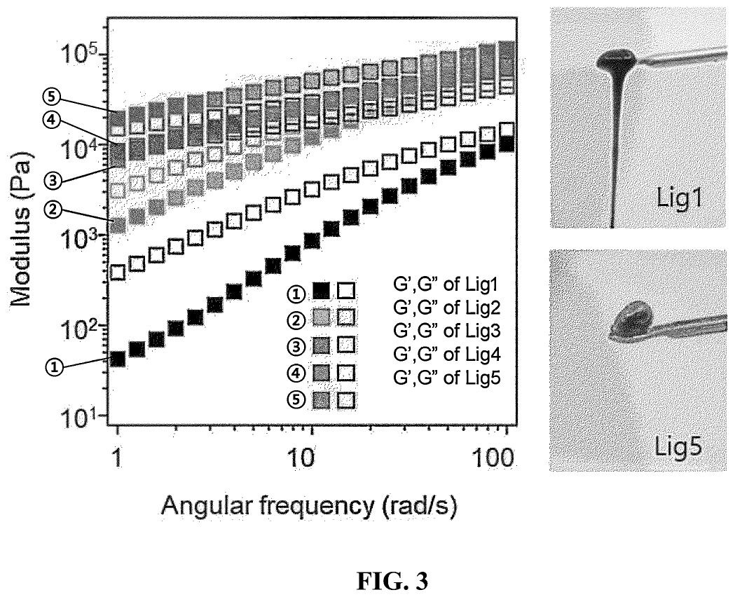

FIG. 3 is a graph illustrating the evaluation results of the mechanical properties of the prepared lignin hydrogel. The viscoelastic property can be controlled by varying the concentrations of lignin and monomers, thereby ensuring similar mechanical properties according to biological tissues. It was found that Lig1 which is a low concentration sample, and Lig5 which is a high concentration sample exhibited significantly different viscoelastic properties.

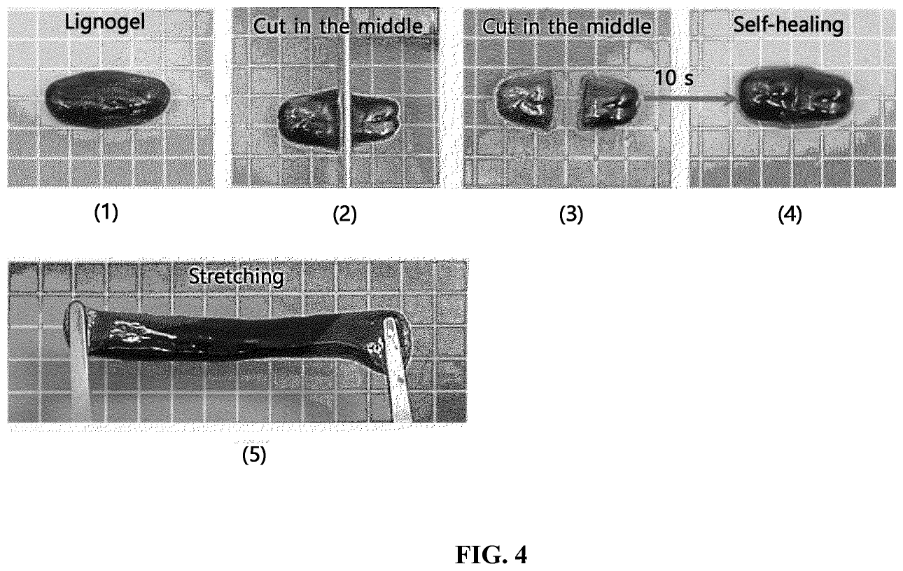

FIG. 4 shows the self-healing properties of lignin hydrogel. It was found that self-healing was performed even after amputation, and mechanical properties were maintained thereafter.

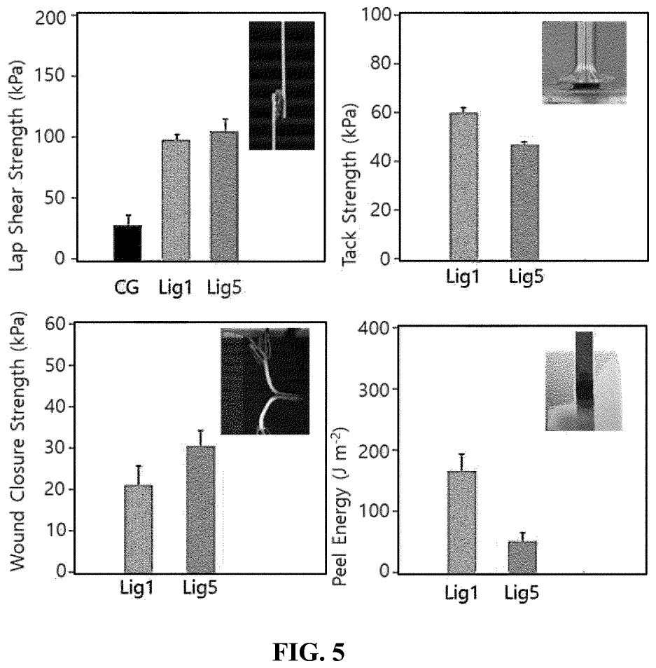

FIG. 5 is a graph illustrating the evaluation results of adhesive strength. Adhesion may be provided through an interaction between functional groups present in lignin hydrogel and functional groups present in skin, and the results of actual adhesion experiments showed adhesive strength that was at least 5-fold higher than that of a conventional bioadhesive (CG).

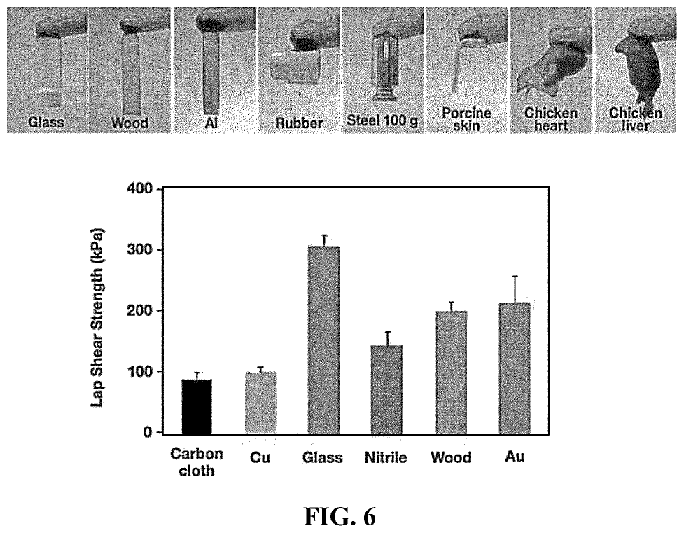

FIG. 6 shows that high adhesive strength is exhibited for various subjects, in addition to those exhibited when targeting living organisms.

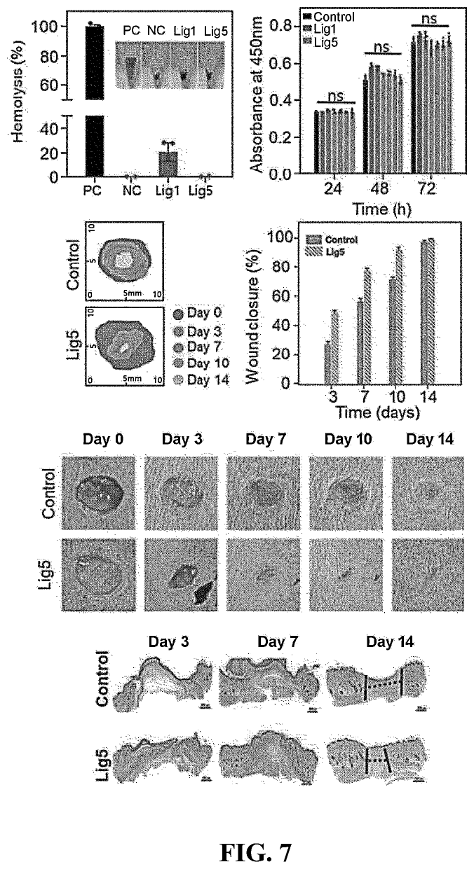

FIG. 7 shows the evaluation results of the possibility whether the lignin hydrogel according to the invention can be included in a bioimplant device. It was confirmed in the examples that there was no hemolysis in the lignin hydrogel, and it was also confirmed from in vitro cytotoxicity test results that the lignin hydrogel was not cytotoxic. In addition, it was confirmed that the wounds to which lignin hydrogel was applied were healed faster than that of the control group.

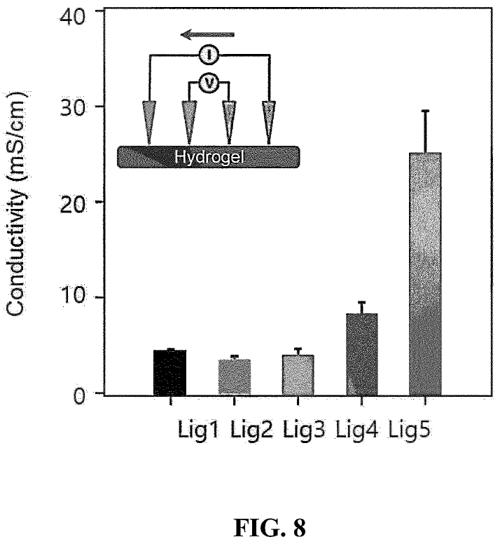

FIG. 8 shows the results of the conductivity of lignin hydrogel through four-terminal resistance measurements. It was found that as the lignin content increased, the electrical conductivity increased. From the results, it is expected that the lignin hydrogel can be used in medical devices, sensors for nerve stimulation, etc.

According to the invention, it is possible to provide an eco-friendly adhesive including lignin hydrogel having excellent biocompatibility and adhesive property.

Additionally, through the method for preparing lignin hydrogel provided in the invention, which is simple and convenient and enables synthesis on a large scale, it is possible to prepare lignin hydrogel effectively and economically, thereby preparing an eco-friendly adhesive through the same.

What is claimed is:

1. An eco-friendly adhesive comprising lignin hydrogel,
wherein said lignin hydrogel comprises a network structure through a bonding between a lignin-based material and a zwitterionic polymer; and
said lignin-based material comprises either one or both of lignin and lignocellulose.

2. The eco-friendly adhesive according to claim 1,
wherein said zwitterionic polymer is any one among 2-(methacryloyloxy)-3-(trimethylammonio) propane-1-sulfonate, 3-pyridinio-2-methacryloyloxypropane-1-sulfonate, arginine methacrylate, 3-N-methylpyrrolidinio-2-methacryloyloxypropane-1-sulfonate, poly(carboxybetaine) based on vinylimidazole, and poly(sulfobetaine) based on vinylimidazole.

3. The eco-friendly adhesive according to claim 1,
wherein said zwitterionic polymer is physically cross-linked by a cation-π interaction and a dipole-dipole interaction on the surface of the lignin-based material.

4. The method according to claim 1,
wherein when the total weight of said lignin hydrogel is 100 parts by weight, the sum of the weights of the lignin-based material and the zwitterionic polymer is 10-70 parts by weight.

5. The eco-friendly adhesive according to claim 4,
wherein when the total weight of said lignin hydrogel is 100 parts by weight, the sum of the weights of the lignin-based material and the zwitterionic polymer is 20-50 parts by weight.

6. The eco-friendly adhesive according to claim 4,
wherein when the total weight of said lignin hydrogel is 100 parts by weight, the sum of the weights of the lignin-based material and the zwitterionic polymer is 10-20 parts by weight; and
said lignin hydrogel has a viscoelastic property.

7. The eco-friendly adhesive according to claim 1,
wherein said eco-friendly adhesive is a bioadhesive.

8. The eco-friendly adhesive according to claim 1,
wherein said bioadhesive is included in a bioimplant device.

9. A method for preparing lignin hydrogel for an eco-friendly adhesive, comprising:
adding lignin or lignocellulose, a zwitterionic monomer, and a polymerization initiator to water and mixing to prepare a mixture; and
irradiating a microwave to said mixture.

10. The method according to claim 9,
wherein said zwitterionic monomer is imidazolium sulfobetaine.

11. The method according to claim 10,
wherein said polymerization initiator is 2,2′-azobis(2-methylpropionamidine) dihydrochloride.

12. The method according to claim 9,
wherein when the total weight of said mixture is 100 parts by weight, the sum of the weights of the lignin-based material and the zwitterionic polymer is 10-70 parts by weight.

13. The method according to claim 9,
wherein said microwave irradiation is performed so that the temperature of the mixture is 60° C. or higher and maintained thereat for 1 hour or more.
